(19) United States
(12) Patent Application Publication
(10) Pub. No.: US 2018/0065125 A1
(43) Pub. Date: Mar. 8, 2018

(54) COMPOSITIONS AND METHODS FOR SEPARATING FLUIDS

(71) Applicant: Momentive Performance Materials Inc., Waterford, NY (US)

(72) Inventor: Srinivas Komati, Suryapet (IN)

(21) Appl. No.: 15/551,941

(22) PCT Filed: Feb. 19, 2016
(86) PCT No.: PCT/US16/18631

§ 371 Date: Aug. 18, 2017

Related U.S. Application Data
(60) Provisional application No. 62/118,529, filed on Feb. 20, 2015.

(51) Int. Cl. B03C 1/00; B03C 1/01; B03C 1/28; C10G 33/02; C01G 49/02; C08G 77/50

(52) U.S. Cl. CPC B03C 1/002; B03C 1/01; B03C 1/288; C07C 53/126; C01G 49/02; C08G 77/50; C01P 2004/64; C10G 33/02

(57) ABSTRACT

A method of separating a fluid containing oil and water into an oil phase and a water phase comprising contacting the fluid with supported magnetic nanoparticles. The supported magnetic nanoparticles can be recovered after being used in a separation process to conduct subsequent separation operations on a fluid.

### CROSS-REFERENCE TO RELATED APPLICATIONS

The present application claims priority to and the benefit of U.S. Provisional Application No. 62/118,529 titled “Compositions and Methods for Separating Fluids,” filed on Feb. 20, 2015, the disclosure of which is incorporated herein by reference in its entirety.

### FIELD

The present invention provides compositions and methods for separating fluids. In particular, the present invention provides compositions and processes for separating fluids using magnetic metal nanoparticles. The magnetic metal nanoparticles may be used to separate fluids and can be used, for example, to separate water from emulsions of water and oil.

### BACKGROUND

Emulsions of water and oil may form during the extraction, production, processing, and/or refining of oils such as, for example, crude oils and petroleum oil. An emulsion is a heterogeneous liquid comprising two liquids in which one of the liquids is intimately dispersed, usually in the form of droplets, in the other liquid. In crude oil emulsions, water is typically dispersed as droplets in the oil, and is referred to as a water-in-oil emulsion.

It may be necessary to separate the water from the oil for further processing or refining of the oil. Separating the water from an oil in an emulsion, which may be referred to as demulsificaiton, may be accomplished by chemical and/or mechanical techniques. Conventional demulsifiers may include, for example, one or more surfactants dissolved in a solvent. Non-ionic surfactants are often used including those comprising polyethylene oxide and propylene oxide groups. Suitable surfactants may include alcohols, fatty acids, fatty amines, fatty esters, glycols, etc. Silicone based demulsifiers are also widely used. Many chemical demulsifiers are slow diffusion demulsifiers and typically cannot be recovered or reused. Many chemical demulsifiers may also not be environmentally friendly.

Mechanical separation may be employed with the use of chemical demulsifiers. For example, electrodemulsification and/or gravity settling and centrifugation may be employed with chemical demulsifiers.

There is still an interest in identifying demulsifiers that may be used to effectively separate fluids and which may provide an environmentally friendly alternative to conventional chemical demulsifiers.

### SUMMARY

The present invention provides compositions and methods for separating fluids such as, for example, emulsions comprising oil and water mixtures. The method comprises adding supported magnetic metal particles to an emulsion. The supported magnetic metal particles cause the emulsion to separate into two phases that can be removed or separated from one another. The supported magnetic particles may be recovered and used for subsequent separation operations.

In one aspect, the present invention provides supported magnetic nanoparticles comprising a metal-containing matrix covalently bonded to a support material.

In one aspect, the present invention provides, a method of separating a composition containing oil and water, the method comprising contacting a composition containing oil and water with supported magnetic nanoparticles to cause the oil and water to be at least partially separated into an oil phase and a water phase, where the supported magnetic nanoparticles comprise functionalized nanoparticle bonded to a support.

In one embodiment, the functionalized nanoparticles comprise a magnetic metal chosen from iron, cobalt, nickel, manganese, or a combination of two or more thereof. In one embodiment, the functionalized nanoparticles comprise particles chosen from Fe₃O₄, Fe₂O₃, Fe₂TiO₄, CoPt, fcc phase FePt, fct phase FePt, FeCo, MnAl, MnBi, Ni₃Fe, FeS, CoFe₂O₄, MnFe₂O₄, or a combination of two or more thereof.

In one embodiment, the functionalized nanoparticles are functionalized by stabilization of the nanoparticles with a C₇-C₃₀ organic fatty acid. In one embodiment, the fatty acid is chosen from lauric acid, oleic acid, stearic acid, myristic acid, hexadecanoic acid, palmitic acid, or a combination of two or more thereof.

In one embodiment, the functionalized nanoparticles comprise magnetic nanoparticles encapsulated in a polymer matrix. In one embodiment, the polymer matrix is chosen from an organic polymer matrix or a siloxane polymer matrix.

In one embodiment, the polymer matrix comprises an organic polymer matrix comprising a polymer or copolymer of a vinyl aromatic, a vinyl halide, an alpha monoolefin, an acrylonitrile, an acrylate, an amide, an acrylamide, an ester, or a combination of two or more thereof.

In one embodiment, the polymer matrix is derived from a silicon hydride-containing polyorganohydrosiloxane of the general formula:

M¹ₐM²_{b}D¹_{c}D²_{d}T¹ₑT²_{f}Qⱼ

wherein: M¹=R¹R²R³SiO_{1/2}; M²=R⁴R⁵R⁶SiO_{1/2}; D¹=R⁷R⁸SiO_{2/2}; D²=R⁹R¹⁰SiO_{2/2}; T¹=R¹¹SiO_{3/2}; T²=R¹²SiO_{3/2}; Q=SiO_{4/2}; R¹, R², R³, R⁴, R⁵, R⁶, R⁷, R⁸, R⁹, R¹⁰, R¹¹, and R¹² are aliphatic, aromatic or fluoro monovalent hydrocarbon having from 1 to 60 carbon atoms; at least one of R⁴, R⁹, R¹² is hydrogen; and the subscript a, b, c, d, e, f, and j are zero or positive subject to the following limitations: 2≦a+b+c+d+e+f+j≦6000, and b+d+f>0.

In one embodiment, the polymer matrix comprises a functional group chosen from a hydride; a carboxyl group, an alkoxy functional group, an epoxy functional group, a triaz-1-yn-2-ium functional group, an anhydride group, a mercapto group, an acrylate, an alkyl, an olefinic, a dienyl, or a combination of two or more thereof.

In one embodiment, the polymer matrix comprises a functional group chosen from —Si—H; —Si(CH₂)ₙCOOR¹³, —Si(CH₂)nSi(OR¹⁴)₃, —Si(OR¹⁵)₁₋₃, —Si(CH₂)ₙ-epoxy, —Si—(CH₂)ₙ-N—N≡N, etc., where R¹³, R¹⁴ and R¹⁵ are independently chosen from hydrogen, hydrocarbyl, substituted hydrocarbyl, or a combination of two or more thereof, and n is chosen from 1 to 26.

In one embodiment, the polymer matrix comprises a polysiloxane. In one embodiment, the polysiloxane is formed from a hydrosiloxane and a vinyl silicon compound.

In one embodiment, the metal-containing polymer matrix has a ratio of polymer to metal of from about 1:1000 to about 100:1; from about 1:1 to about 20:1; from about 10:1 to about 20:1; even from about 12:1 to about 16:1.

In one embodiment, the metal particles have a particle size of from about 1 to about 100 nanometers.

In one embodiment, the support material is chosen from silicon, a silicate such as a sodium silicate, a borosilicate, or a calcium aluminum silicates, clay, silicate, silica, starch, carbon, alumina, titanic, calcium carbonate, barium carbonate, zirconia, metal oxide, carbon nanotubes, synthetic and natural zeolites, polymeric resins in bead or fibrous form, or and mixtures of two or more thereof.

In one embodiment, the metal loading ranges from about 0.001 to 20 percent by weight of the support material; from about 0.05 to about 5 percent by weight of the support material; even from about 0.1 to about 1 percent by weight of the support material.

In one embodiment, the support material comprises a functional group chosen from a group such as silanol, alkoxy, acetoxy, silazane, oximino-functional silyl group, hydroxyl, acyloxy, ketoximino, amine, aminoxy, alkylamide, hydrogen, allyl, an aliphatic olefinic group, aryl, hydrosulfide, or a combination of two or more thereof.

In one embodiment, the support material comprises a functional group chosen from —Si—CH═CH₂, —Si—OH, —Si—(CH₂)ₙC≡CH, —Si—(CH₂)ₙ—NH₂, —Si—(CH₂)ₙ—OH, —Si—(CH₂)ₙ—SH, or a combination of two or more thereof, and n is 1-26, 1-10, even 1-8.

In one embodiment, the metal-containing polymer matrix is covalently bonded to the support material via a hydrophobic functional group attached to the support material. In one embodiment, the hydrophobic group is chosen from an alkyldisilazane, a vinyl-containing silazane, or a combination thereof

In one embodiment, the composition containing oil and water is at a temperature of from about 1° C. to about 1000° C.

In one embodiment, the method further comprises applying a magnetic field to the water phase and removing the oil phase from the composition.

In one embodiment, the step of removing the oil phase comprises decanting the oil phase from the composition.

In one embodiment, the method further comprises removing the supported magnetic particles from the water phase.

In one embodiment, the method further comprises washing the supported magnetic particles that were removed from the water phase.

In one embodiment, the method comprises reusing the supported magnetic particles that were removed from the water phase in a subsequent operation to separate a composition comprising oil and water.

In one embodiment, the composition is a water-in-oil emulsion.

In one embodiment, the composition is an oil-in-water emulsion.

In one embodiment, the oil is chosen from a crude oil, a crude oil distillate, bitumen, a crude oil-light oil blend, a vegetable oil, an animal oil, a synthetic oil, or a combination of two or more thereof

In another aspect, the present in provides a solid demulsifier comprising functionalized magnetic metal nanoparticles covalently bonded to a support, wherein the functionalized magnetic nanoparticles comprise nanoparticles that exhibit magnetic properties, and said nanoparticles are encapsulated in a polymer matrix and/or are functionalized by stabilization of the nanoparticles with a C₇-C₃₀ organic fatty acid.

In still another aspect, the present invention provides an emulsion comprising an oil, water, and a solid demulsifier, wherein the solid demulsifier comprises functionalized magnetic metal nanoparticles covalently bonded to a support, wherein the functionalized magnetic nanoparticles comprise nanoparticles that exhibit magnetic properties.

In one embodiment of the emulsion, the nanoparticles are encapsulated in a polymer matrix and/or are functionalized by stabilization of the nanoparticles with a C₇-C₃₀ organic fatty acid.

In one embodiment of the emulsion, the oil is chosen from a crude oil, a crude oil distillate, bitumen, a crude oil-light oil blend, a vegetable oil, an animal oil, a synthetic oil, or a combination of two or more thereof

These and other aspects and embodiments of the invention are further understood with reference to the detailed description.

### DETAILED DESCRIPTION

The present invention provides compositions and methods for separating fluids using supported magnetic nanoparticles. In one embodiment, the supported magnetic nanoparticles comprise a metal-containing matrix bonded to a support material. The metal-containing matrix comprises a polymer matrix having a plurality of metal nanoparticles disposed in the matrix. The support material comprises a substrate having functional groups at or near the substrate's surface that are capable of bonding with the metal-containing matrix material. The supported magnetic nanoparticles may be employed as a demulsifying agent to separate and/or remove water from an emulsion.

### Metal Nanoparticles

The demulsifying agent comprises magnetic metal nanoparticles bonded to a support. The magnetic metal nanoparticles are functionalized to allow for attachment of the particles to the support. The magnetic metal nanoparticles may be functionalized by a matrix material coating and/or stabilizing the nanoparticles. The nanoparticles can be coated and/or stabilized with a matrix material comprising a ligand or functional group to allow for bonding to the support.

In one aspect, the present invention stabilizes metal particles sterically through coating with a fatty acid such as, for example, lauric acid, and simultaneously anchors the resulting fatty acid coated metal particles to silica particles via covalent bonding linkages. In one embodiment, the matrix for coating and/or stabilizing the particles is chosen from one or more fatty acids. In one embodiment, the fatty acid may be a C₇-C₃₀ organic fatty acid. Examples of suitable fatty acids include, but are not limited to, lauric acid, oleic acid, stearic acid, myristic acid, hexadecanoic acid, palmitic acid, etc., or a combination of two or more thereof

Other suitable materials for coating and/or stabilizing the particles include, but are not limited to, ionic salts. Ionic salts may include, for example, hydroxides, nitrates, oxalates, acetates, phophates, carbonates, etc., of ammonium, phosphonium, sulfonium, etc. In an embodiment, the salt for the matrix/coating is chosen from one or more lower alkyl quaternary ammonium salts. Examples of suitable ammonium salts include tetramethylammonium hydroxide (TMAH), tetraethylammonium hydroxide, tetrapropylammonium hydroxide, trimethylethylammonium hydroxide, (2-hydroxyethyl)trimethylammonium hydroxide, (2-hydroxyethyl)triethylammonium hydroxide, (2-hydroxyethyl)tripropylammonium hydroxide, (1-hydroxypropyl)trimethylammonium hydroxide, tetramethylphosphonium hydroxide, tetraethylphosphonium hydroxide, tetrapropylphosphonium hydroxide, tetrabutylphosphonium hydroxide, trimethylhy droxy ethylphosphonium hydroxide, dimethyldihy droxy ethylphosphonium hydroxide, methyltrihy droxy ethylphosphonium hydroxide, phenyltrimethylphosphonium hydroxide, phenyltriethylphosphonium hydroxide and benzyltrimethylphosphonium hydroxide, trimethylsulfonium hydroxide, triethylsulfonium hydroxide, tripropylsulfonium hydroxide, etc., or combinations of two or more thereof.

The matrix and/or coating for the nanoparticles may also be a polymer material. Examples of suitable polymer materials include, but are not limited to, hydrophilic materials such as, but not limited to, polyalkylene glycols, polyvinyl alcohols, polyacrylic acid, cellulosic materials, polyalkylene imides, polyoxyalkylenes, polyvinyl pyrrolidones, etc. Non-limiting examples of suitable polymeric coatings include, but are not limited to, polyethylene glycol, polypropylene glycol, polyoxyethylene, poly(N-vinyl-2-pyrrolidone), dextran, etc., or a combination of two or more thereof.

In one embodiment, the magnetic nanoparticles are provided as a magnetic metal-containing polymer matrix having a polymer matrix comprising a plurality of magnetic metal nanoparticles dispersed in the polymer matrix. In an embodiment, the magnetic metal nanoparticles are encapsulated in the polymer matrix. The polymer matrix may be selected as desired for a particular purpose or intended use. For example, the polymer matrix may be chosen to provide a particular functionality for bonding with the substrate material or based on the environment in which the demulsifier will be used.

Metal nanoparticles may be stabilized by the combination of steric and electrostatic stabilization. In one embodiment, the matrix and/or coating for the nanoparticles may also be a combination of polymer surfactants and ionic surfactants.

In one embodiment, the polymer matrix can comprise one or more organic synthetic polymer materials. Suitable organic synthetic polymer materials include, but are not limited to thermoplastic polymers, thermoplastic elastomers, etc. Suitable organic polymer materials can include polymers or copolymers of vinyl aromatic monomers, such as styrene; vinyl halide such as vinyl chloride; acrylonitrile; alpha-monoolefins such as ethylene, propylene, etc.; acrylates; acrylamides; amides; esters; etc., or a combination of two or more thereof

In one embodiment, the polymer matrix may comprise a cross linked polysiloxane network. The polysiloxane network can comprise a crosslinked or partially crosslinked network of hydrosiloxanes or hydride fluid with a vinyl silicon compound. In one embodiment, the hydrosiloxanes are polyorganohydrosiloxanes comprising a silicon hydride (Si—H) group. In one embodiment, the polyorganohydrosiloxane is of Formula (1):

M¹ₐM²_{b}D¹_{c}D²_{d}T¹ₑT²_{f}Qⱼ. (1)

wherein: M¹=R¹R²R³SiO_{1/2}; M²=R⁴R⁵R⁶SiO_{1/2}; D¹=R⁷R⁸SiO_{2/2}; D²=R⁹R¹⁰SiO_{2/2}; T¹=R¹¹SiO_{3/2}; T²=R¹²SiO_{3/2}; Q=SiO_{4/2}; R¹, R², R³, R⁴, R⁵, R⁶, R⁷, R⁸, R⁹, R¹⁰, R¹¹, and R¹² are independently aliphatic, aromatic, cycloaliphatic, or fluoro monovalent hydrocarbon having from 1 to 60 carbon atoms, and at least one of R⁴, R⁹, and R¹² is hydrogen. Examples of useful aliphatic groups include, but are not limited to, methyl, ethyl, n-propyl, iso-propyl, n-butyl, isobutyl, tert-butyl, n-pentyl, iso-pentyl, neopentyl and tert-pentyl; hexyl, such as the n-hexyl group; heptyl, such as the n-heptyl group; octyl, such as the n-octyl, isooctyl groups, and the 2,2,4-trimethylpentyl group; nonyl, such as the n-nonyl group; decyl, such as the n-decyl group; cycloalkyl radicals, such as cyclopentyl, cyclohexyl and cycloheptyl radicals and methylcyclohexyl radicals. Examples of suitable aryl groups include, but are not limited to, phenyl, naphthyl; o-, m- and p-tolyl, xylyl, ethylphenyl, and benzyl. R⁴, R⁹, R¹² are independently selected from hydrogen. The subscripts a, b, c, d, e, f, and j are zero or positive subject to the following limitations: 2≦a+b+c+d+e+f+j≦6000, b+d+f>0. The Si—H content of the polysiloxanes can range from about 0.001 to about 99 mole percent; about 0.01 to about 95 mole percent; about 0.1 to about 90 mole percent; about 1 to about 75 mole percent; about 5 to about 50 mole percent; even about 10 to about 25 mole percent.

The polysiloxane can comprise a variety of functionalities to allow the metal-containing polymer matrix to be bonded or adhered to the support material. Examples of suitable functional groups include, but are not limited to, hydride functionalities (—SiH); carboxyl functional groups, alkoxy functional groups, epoxy functional groups, a triaz-1-yn-2-ium functional group, an anhydride group, a mercapto group, an acrylate, an alkyl, olefinic, dienyl, etc. or a combination of two or more thereof. Non-limiting examples of suitable functional groups include —Si—H; —Si(CH₂)ₙCOOR¹³, —Si(CH₂)nSi(OR¹⁴)₃, —Si(OR¹⁵)₁₋₃, —Si(CH₂),-epoxy, —Si—(CH₂)ₙN—N≡N, etc. where R¹³, R¹⁴, and R¹⁵ can be hydrogen, hydrocarbyl, substituted hydrocarbyl, or a combination of two or more thereof, and n can be 1 to 26, 2 to 10, even 2 to 8. In one embodiment, the functional group is a —Si(CH₂)ₙCOOR¹³ group, where R¹³ is hydrogen, and n is 1 to 26, in another embodiment 5 to 20, and in yet another embodiment 7 to 15.

In one embodiment, the polymer matrix comprises a polyalkyl hydrosiloxane, a polyaryl hydrosiloxane, or a combination of two or more thereof. In one embodiment, the polymer matrix comprises a hydrosiloxane chosen from poly(methyl hydrosiloxane) (PMHS), poly(ethyl hydrosiloxane), poly(propyl hydrosiloxane), polyaryl hydrosiloxane (e.g., poly(phenyl hydrosiloxane), poly(tolyl hydrosiloxane)), poly(phenyl dimethylhydrosiloxy)siloxane, poly(dimethyl siloxane co-methyl hydrosiloxane), poly(methyl hydrosiloxane co-phenyl methyl siloxane), poly(methyl hydrosiloxane coalkyl methyl siloxane), poly(methyl hydrosiloxane co-diphenyl siloxane), poly(methyl hydrosiloxane co-phenyl methyl siloxane). The hydrosiloxane can be a homopolymer or a copolymer comprising two or more hydrosiloxanes.

The vinyl silicon compound is not particularly limited and can be, for example, a cyclic vinyl siloxane, a non-cyclic vinyl siloxane, or a combination of two or more thereof. Examples of suitable vinyl siloxanes includes, but are not limited to, 1,3-divinyl-1,1,3,3-tetramethyl disoloxane, 1,3,5 trimethyl-1,3,5-trivinyl-cyclotrisiloxane, 1,3,5,7-tetramethyl- 1,3,5,7-tetravinyl-cyclotetrasiloxane, etc.

The weight average molecular weight of the polysiloxanes of the present invention can range from about 150 to about 50000, about 200 to about 30000, about 250 to about 25000, about 300 to about 15000, even about 500 to about 10000. Here as elsewhere in the specification and claims, numerical values can be combined to form new and non-disclosed ranges. It will be appreciated that the polysiloxane network may have some residual hydride bonds.

The metal nanoparticle material may be chosen as desired for a particular purpose or intended use. In accordance with the present invention, the metal-containing polymer matrix comprises nanoparticles chosen from magnetic nanoparticles. The magnetic nanoparticles include materials exhibiting magnetic properties. The magnetic nanoparticles can have various magnetic properties including, but not limited to, diamagnetic, paramagnetic, supermagnetic, ferromagnetic, antiferromagnetic, spin glass, electromagnetic, etc. Suitable metals for the magnetic nanoparticles include, but are not limited to, cobalt, iron, manganese, nickel, or a combination of two or more thereof The magnetic nanoparticles may comprise other metals including, but not limited to, aluminum, iron, silver, zinc, gold, copper, platinum, rhodium, ruthenium, palladium, titanium, vanadium, chromium, molybdenum, cadmium, mercury, calcium, zirconium, iridium, cerium, oxides and sulfides of such metals, or a combination of two or more nanoparticles thereof. In one embodiment, the nanoparticles comprise alloys of two or more metals. The magnetic nanoparticles may comprise one, two, three, or more metals. In one embodiment, the metal nanoparticles comprise iron. Examples of suitable magnetic metal nanoparticles include, but are not limited to, particles of Fe₃O₄, Fe₂O₃, Fe₂TiO₄, CoPt, fcc phase FePt, fct phase FePt, FeCo, MnAl, MnBi, Ni₃Fe, FeS, CoFe₂O₄, MnFe₂O₄, combinations of two or more thereof, etc.

The magnetic metal nanoparticles may be any suitable nanostructure. In one embodiment, the nanoparticles are chosen from nanospheres, nanocubes, nanotubes, nanorods, etc., or combinations of two or more thereof.

In one embodiment, the metal nanoparticles have a particle size of from about 1 to about 100 nanometers (nm). In another embodiment, the metal nanoparticles have a particle size of from about 5 to about 90 nanometers. In still another embodiment, the metal nanoparticles have a particle size of from about 10 to about 80 nanometers. In yet another embodiment, the metal nanoparticles have a particle size of from about 20 to about 70 nanometers (nm). In an even further embodiment, the metal nanoparticles have a particle size of from about 30 to about 60 nanometers (nm). In yet a further embodiment, the metal nanoparticles have a particle size of from about 35 to about 50 nanometers (nm). Here as elsewhere in the specification and claims, numerical values may be combined to form new or undisclosed ranges. The particle size of the metal nanoparticles may be determined by any suitable method. In one embodiment, particle size is determined by transmission electron microscopy (TEM).

In one embodiment, the weight ratio of the polymer matrix to metal is from about 1:1000 to about 100:1. In another embodiment, the weight ratio of polymer to metal is from about 1:100 to about 100:1. In another embodiment, the weight ratio of polymer to metal is from about 1:50 to about 50:1. In another embodiment, the weight ratio of polymer to metal is from about 1:10 to about 50:1. In another embodiment, the weight ratio of polymer to metal is from about 1:1 to about 35:1. In another embodiment, the weight ratio of polymer to metal is from about 1:1 to about 20:1. In another embodiment, the weight ratio of polymer to metal is from about 10:1 to about 20:1. In another embodiment, the weight ratio of polymer to metal is from about 12:1 to about 16:1. In one embodiment, the weight ratio of polymer to metal is about 15:1. Here as elsewhere in the specification and claims, numerical values may be combined to form new or nondisclosed ranges.

The metal-containing polymer matrix may be formed by reducing a metal complex in solution to form metal nanoparticles, metal oxide nanoparticles, or metal sulfide nanoparticles. In one embodiment, the solution for reducing the metal complex also serves as the polymer material for forming the polymer matrix. In one embodiment, the solution for reducing the metal complex is a silicon hydride containing polyorganohydrosiloxane. Non-limiting examples of suitable polyorganohydrosiloxane materials can be those described above.

In one embodiment, the method for forming the metal-containing polymer matrix comprises reacting a metal complex with a silicon hydride containing polyorganohydrosiloxane solution in a suitable solvent to form a colloidal suspension of metal nanoparticles and subsequently reacting the suspension to form a polymer matrix. The reaction may be carried out in an inert atmosphere, such as under a nitrogen atmosphere, to form the metal nanoparticles. In one embodiment, the reaction to form the metal nanoparticles is carried out at a temperature of about 80° C. Following formation of the nanoparticles, the suspension is subjected to an oxygen environment to effect polymerization and encapsulate the metal nanoparticles. The reaction in the presence of oxygen can be carried out for a period of from about 5 to about 40 minutes, in one embodiment from about 10 to about 30 minutes, in another embodiment, from about 15 to about 25 minutes.

The method can also comprise removing an amount of solvent from the colloidal suspension prior to the polymerization/encapsulation reaction. In one embodiment, at least about 50% of the initial solvent content is removed; in another embodiment at least about 60% of the initial solvent content is removed; in another embodiment, at least about 70% of the initial solvent content is removed; in another embodiment, at least about 80% of the initial solvent content is removed. In one embodiment, about 50% to about 100% of the initial solvent content is removed; in another embodiment about 60% to about 100% of the initial solvent content is removed; in another embodiment, about 70% to about 100% of the initial solvent content is removed; in another embodiment about 80% to about 100% of the initial solvent content is removed

The metal complex for forming the metal nanoparticles can be a metal compound suitable for providing the desired metal. The metal complex can be a metal compound comprising a magnetic metal chosen from iron, cobalt, nickel, magnesium, or a combination of two or more thereof. Other metals may be employed to provide a desired magnetic particle including, but not limited to aluminum, silver, zinc, gold, copper, platinum, rhodium, ruthenium, palladium, titanium, vanadium, chromium, molybdenum, cadmium, mercury, calcium, zirconium, iridium, cerium, or a combination of two or more thereof. Examples of suitable metal complexes for forming metal nanoparticles include, but are not limited to, FeCl₂.6H₂O, CoCl₂.6H₂O, NiCl₂.6H₂O, MnCl₂.4H₂O, PtCl₂, H₂PtCl₆, Pt₂(dba)₃, Pt₂(dvs)₃, Pt(OAc)₂ Pt(acac)₂,Na₂PtCl₆, K₂PtCl₆, platinum carbonate, platinum nitrate, 1,5-cyclooctadienedimethylplatinum(II), platinum perchlorate, amine complexes of the platinum, ammonium hexachloropalladate(IV), palladium(II) chloride, AuCl₃, Au₂O₃, NaAuO₂, AgCl, AgNO₃, CuSO₄, CuO, CU(NO₃)₂, CUCl₂, Ru₂O₃, RUCl₂, ZnCl₂, TiCl₄, vanadium chloride, cadmium chloride, calcium chloride, zirconium tetrachloride, mercuric chloride complexes. As used herein, “dba” refers to dibenzylideneacetone, “dvs” refers to divinyl tetramethyl disiloxane, “OAc” refers to acetate anion, and “acac” refers to acetylacetone ligand.

The support material can be selected as desired for a particular purpose or intended use. In one embodiment, the support material can be organic polymer material, an inorganic material, etc. Examples of suitable support materials include, but are not limited to, silicon, silicates such as sodium silicates, borosilicates or calcium aluminum silicates, different types of clay, silica, starch, carbon, alumina, titania, calcium carbonate, barium carbonate, zirconia, metal oxide carbon, nanotubes, synthetic and natural zeolites, polymeric resins in bead or fibrous form, or mixtures of two or more thereof. Examples of suitable organic materials include, polymers containing unsaturated functional groups such as styrene or vinyl containing compounds. Other examples of suitable organic resins include sulfonate resins such as Nafion® resin available from DuPont.

The support material can generally be provided as particles. In one embodiment, the support particles have a particle size of from about 50 to about 1000 micrometers. In one embodiment, the support particles have a particle size of from about 100 to about 800 micrometers. In one embodiment, the support particles have a particle size of from about 200 to about 700 micrometers. In one embodiment, the support particles have a particle size of from about 300 to about 600 micrometers. Here as elsewhere in the specification and claims, numerical values may be combined to form new or nondisclosed ranges. The particle size of the support particles may be determined by any suitable method. In one embodiment, particle size is determined by scanning electron microscopy (SEM).

The support material comprises a functional group attached thereto that is capable of reacting with a moiety of the polymer matrix such that the metal-containing polymer matrix is chemically bonded to the support material. The support material may be hydrophilic or hydrophobic. It will be appreciated that the functional group can be provided by the natural surface characteristics of the particles (e.g., surface OH groups on silica) or the particles may be functionalized with a selected moiety to provide a desired reactive site or reactivity. In one embodiment, where the polymer matrix contains a hydrosilane (SiH) moiety, the support material can be functionalized with any group that can react with the SiH moiety such as, for example, via a hydrosilylation reaction, a condensation reaction, etc. In one embodiment, the support material can be modified with a compound comprising a functional group chosen from a group such as silanol, alkoxy, acetoxy, silazane, oximino-functional silyl group, hydroxyl, acyloxy, ketoximino, amine, aminoxy, alkylamide, hydrogen, allyl or other aliphatic olefinic group, aryl, hydrosulfide, a combination of two or more thereof etc. Silanol, alkoxy, and acetoxy groups are all capable of condensing with Si—H groups. In one embodiment, the support material comprises a functional group having an unsaturated carbon-carbon bond (e.g., a double bond or a triple bond). In one embodiment, the support material has a functional group chosen from —Si—CH═CH₂, —Si—OH, —Si—(CH₂)ₙC≡CH, —Si—(CH₂)ₙ—NH₂, —Si—(CH₂)ₙ—OH, —Si—(CH₂)ₙ—SH, a combination of two or more thereof, etc, and n 1-26, 1-10, even 1-8. The functional groups provided on the support material can be chosen as desired to facilitate bonding with the functional groups provided on the polymer matrix of the metal-containing matrix material to bond or anchor the metal-containing polymer matrix to the support.

In the case of silica support particles and a metal-containing matrix comprising a hydrosiloxane polymer, the inventors have found that it may be beneficial to functionalize the silica particles with a hydrophobic group to facilitate reaction with the hydrophobic siloxane polymer. In the present invention, this specific functionalization process (i.e., treating the material with a hydrophobic group) is referred to as “capping.”

In one embodiment, the substrate particle is functionalized with a silazane. The silazane compound is a generic name of a compound having a Si—N bond in its molecule. Suitable silazanes include, but are not limited to, disilazanes such as alkyldisilazanes. Specific examples of suitable silazanes include, but are not limited to, dimethyl disilazane, trimethyl disilazane, tetramethyl disilazane, pentamethyl disilazane, hexamethyl disilazane (HMDZ), octamethyl trisilazane, hexamethylcyclo trisilazane, tetraethyltetramethylcyclo tetrasilazane, tetraphenyldimethyl disilazane, dipropyltetramethyl disilazane, dibutyltetramethyl disilazane, dihexyltetramethyl disilazane, dioctyltetramethyl disilazane, diphenyl tetramethyl disilazane, and octamethylcyclo tetrasilazane. In addition, a fluorine-containing organic silazane compound obtained by substituting a silazane compound partially with fluorine may be used. In still other embodiments, the silazane compounds comprise carbon-carbon double bonds such as, for example, vinyl groups. An example of a suitable vinyl-containing silazane is divinyltetramethylsilazane (DVTMDZ). Other vinyl-containing compounds useful in the process are vinyltriacetoxysilane and vinyltrialkoxysilanes such as vinyl trimethoxysilane, vinyltriethoxysilane, and vilytriisoproxysilanes.

In one embodiment, the functionalized substrates comprise a combination of alkyldisilazanes and vinyl-containing disilazanes. The weight ratio of alkyldisilazane to vinyl-containing disilazane can be from about 1000:1 to about 1:1000. In one embodiment, the weight ratio of alkyldisilazane to vinyl-containing disilazane can be from about 500:1 to about 1:500. In another embodiment, the weight ratio of alkyldisilazane to vinyl-containing disilazane can be from about 100:1 to about 1:100. In still another embodiment, the weight ratio of alkyldisilazane to vinyl-containing disilazane can be from about 10:1 to about 1:10. Here as elsewhere in the specification and claims, numerical values can be combined to form new and non-disclosed ranges. In one embodiment, the substrates are functionalized with both hexamethyldisilazane and divinyltetramethylsilazane.

The supported magnetic particles comprise a matrix material attached to the (functionalized) substrate particles. The metal-containing matrix material can be formed by reacting the metal-containing matrix material and the substrate under conditions sufficient to bond the matrix material to the functional groups on the substrates. In one embodiment, the metal-containing matrix comprises a polyhydrosiloxane comprising SiH groups, and the SiH groups react with the functional groups disposed on the substrate material.

The reaction of the polymer functional moieties with the functional groups attached to the substrate can be carried out by any suitable means depending on the moieties undergoing reaction. For example, the reaction may be carried out in the presence or absence of a solvent as desired. In one embodiment the reaction is carried out at a temperature of from about 5° C. and 150° C. In one embodiment, the reaction is carried out at a pressure ranging from about 0.001 to about 10 bar.

The metal loading concentration in the metal catalyst material can be from about 0.001 to about 20 percent by weight based on the total weight of the substrate particles. In one embodiment, the metal loading concentration in the metal catalyst material can be from about 0.01 to about 15 percent by weight based on the total weight of the substrate particles. In another embodiment, the metal loading concentration in the metal catalyst material may be from about 0.05 to about 5 percent by weight based on the total weight of the substrate particles. In still another embodiment, the metal loading concentration in the metal catalyst material may be from about 0.1 to about 1 percent by weight based on the total weight of the substrate particles.

### Separation of Fluids

The supported magnetic nanoparticles may be used to separate fluids such as separating an emulsion comprising oil and water. In one embodiment, the supported magnetic nanoparticles may be employed to separate a fluid comprising oil and water into separate phases by contacting the supported magnetic nanoparticles with the fluid. The supported magnetic particles may be added to the solution to be separated in an amount as desired suitable to effectuate the separation of the oil an aqueous phases. In one embodiment, the particles may be added to the solution to be separated in an amount of from about 0.01 to about 5 wt. %; from about 0.05 to about 2.5 wt. %; even from about 0.1 to about 1 wt. % based on the weight of the solution to be separated. In one embodiment, the supported magnetic particles may be added in an amount of from about 0.01 to about 0.05 wt. %. Here as elsewhere in the specification, numerical values may be combined to form new and non-disclosed ranges. In one embodiment, the temperature of the fluid is from about 1° C. to about 1000° C.; from about 25° C. to about 500° C.; from about 50° C. to about 100° C.; even from about 65° C. to about 80° C. Here as elsewhere in the specification, numerical values may be combined to form new and non-disclosed ranges. The phases may be separated by decanting the oil phase off of the water phase. In decanting the oil phase from the water phase, a magnetic force may be applied to the water to hold the water phase separate from the oil phase.

After separation of the oil and water phases, the supported magnetic nanoparticles may be recovered from the water phase. This may be accomplished by filtering the water phase. The supported magnetic nanoparticles may be washed with a suitable solvent to remove any oil residue. The particles may then be reused to treat other fluids. In one embodiment, water may be used to wash the supported magnetic particles.

The supported magnetic nanoparticles may be used to treat a fluid such as an emulsion. In one embodiment, the emulsion is an emulsion comprising water and an oil. In one embodiment, the fluid may be a water-in-oil emulsion. In another embodiment, the fluid may be an oil-in-water emulsion. The oil may generally be any oil comprising one or more condensable hydrocarbons. The oil may be derived from a crude oil, a crude oil distillate, bitumen, a crude oil-light oil blend, a vegetable oil, an animal oil, a synthetic oil, or a combination of two or more thereof. Crude oils may comprise various components including solids, asphaltenes, organic acids, basic nitrogen containing compounds, etc. The emulsions may also include hydrocarbon emulsions derived from refined mineral oil, gasoline, kerosene, etc.

While the invention has been described with respect to various embodiments, aspects of the invention may be further understood in view of the following examples. The examples are for illustrating aspects of the invention and are not intended to limit the invention.

### EXAMPLES

### Example 1: Synthesis of Fe₃O₄/Silica Particles

1 gram of ferrous chloride tetrahydrate (99% FeCl₂.4H₂O, Sigma-Aldrich) was dissolved in 4 mL of 2M HCl solution (Solution A), then 2.719 grams of ferric chloride hexahydrate (97% FeCl₃.6H₂O, Sigma-Aldrich) was dissolved in 10 mL of deionized water (Solution B). Solutions A and B were mixed in a 100 mL beaker (Solution C). Then 13 mL of (25 wt %) ammonia solution (NH₄OH, Merck) was added dropwise to solution C from a burette with vigorous stirring under N₂. This mixture represents an initial molar ratio of Fe (III) to Fe (II) is 2:1. Once the ammonia solution was added completely, the color of the mixture turned to black. Then, 18 mL of lauric acid was added to the suspension and the stabilization of Fe₃O₄ nanoparticles in a Lauric acid is continued for 15 mins. The Lauric acid stabilized Fe₃O₄ nanoparticles (taken out from flask) and 7.5 g hydrophilic silica (particle size: 100-200 mesh size or 80-100 μm) from example 1 are transferred into a petri dish and mixed thoroughly to form a homogeneous demulsifier powder. This solid demulsifier is then further dried in an oven for 3 hrs to remove any volatile contents. This gives a Fe₃O₄/SiO₂ demulsification powder having a Fe₃O₄ content of 0.1% by weight.

### Example 2: Demulsification of Crude

Demulsification of Sobhesan crude oil was performed using bottle tests. The supported magnetic particles of Example 1 were added to a sample of Sobhesan crude oil in an amount of 0.01-0.05 wt. %. The samples were heated to a temperature of 70° C. for one hour. The emulsion separated into two phases, an upper phase of crude oil, and a lower water phase containing the supported magnetic particles. The crude oil phase was decanted off of the water phase.

The supported magnetic particles were recovered by filtration of the water phase. The recovered magnetic particles were washed with water and dried, and used in a subsequent demulsification process.

Embodiments of the invention have been described above and, obviously, modifications and alterations will occur to others upon the reading and understanding of this specification. The invention and any claims are intended to include all modifications and alterations insofar as they come within the scope of the claims or the equivalent thereof.

1. A method of separating a composition containing oil and water, the method comprising:
contacting a composition containing oil and water with supported magnetic nanoparticles to cause the oil and water to be at least partially separated into an oil phase and a water phase, where the supported magnetic nanoparticles comprise functionalized nanoparticle bonded to a support material.

2. The method of claim 1 wherein the functionalized nanoparticles comprise a magnetic metal chosen from iron, cobalt, nickel, manganese, or a combination of two or more thereof.

3. The method of claim 2, wherein the functionalized nanoparticles comprise particles chosen from Fe₃O₄, Fe₂O₃, Fe₂TiO₄, CoPt, fcc phase FePt, fct phase FePt, FeCo, MnAl, MnBi, Ni3Fe, FeS, CoFe₂O₄, MnFe₂O₄, or a combination of two or more thereof

4. The method of claim 2, wherein the functionalized nanoparticles are functionalized by stabilization of the nanoparticles with a C₇-C₃₀ organic fatty acid.

5. The method of claim 4, wherein the fatty acid is chosen from lauric acid, oleic acid, stearic acid, myristic acid, hexadecanoic acid, palmitic acid, or a combination of two or more thereof.

6. The method of claim 1, wherein the functionalized nanoparticles comprise magnetic nanoparticles encapsulated in a polymer matrix.

7. The method of claim 6, wherein the polymer matrix comprises an organic polymer matrix comprising a polymer or copolymer of a vinyl aromatic, a vinyl halide, an alpha monoolefin, an acrylonitrile, an acrylate, an amide, an acrylamide, an ester, or a combination of two or more thereof.

8. The method of claim 6, wherein the polymer matrix is derived from a silicon hydride-containing polyorganohydrosiloxane of the general formula:
M¹ₐM²_{b}D¹_{c}D²_{d}T¹ₑT²_{f}Qⱼ
wherein: M¹=R¹R²R³SiO_{1/2}; M²=R⁴R⁵R⁶SiO_{1/2}; D¹=R⁷R⁸SiO_{2/2}; D²=R⁹R¹⁰SiO_{2/2}; T¹=R¹¹SiO_{3/2}; T²=R¹²SiO_{3/2}; Q=SiO_{4/2}; R¹, R², R³, R⁴, R⁵, R⁶, R⁷, R⁸, R⁹, R¹⁰, R¹¹, and R¹² are aliphatic, aromatic or fluoro monovalent hydrocarbon having from 1 to 60 carbon atoms; at least one of R⁴, R⁹, R¹² is hydrogen; and the subscript a, b, c, d, e, f, and j are zero or positive subject to the following limitations: 2≦a+b+c+d+e+f+j≦6000, and b+d+f>0.

9. The method of claim 6, wherein the polymer matrix comprises a functional group chosen from a hydride; a carboxyl group, an alkoxy functional group, an epoxy functional group, a triaz-1-yn-2-ium functional group, an anhydride group, a mercapto group, an acrylate, an alkyl, an olefinic, a dienyl, or a combination of two or more thereof.

10. The method of claim 6, wherein the polymer matrix comprises a functional group chosen from —Sᵢ—H; —Si(CH₂)ₙCOOR¹³, —Si(CH₂nSi(OR¹⁴)₃, —Si(OR¹⁵)₁₋₃, —Sᵢ(CH₂)ₙ-epoxy, —Si—(CH₂)ₙ—N—N≡N, etc. where R¹³, R¹⁴, and R¹⁵ is chosen from hydrogen, hydrocarbyl, substituted hydrocarbyl, or a combination of two or more thereof, and n is chosen from 1 to 26.

11. The method of claim 10, wherein the polymer matrix comprises a polysiloxane.

12. The method of claim 11, wherein the polysiloxane is formed from a hydrosiloxane and a vinyl silicon compound.

13. The method of claim 6 wherein the polymer matrix has a ratio of polymer to metal of from about 1:1000 to about 100:1.

14. The method of any of claims 1 wherein the nanoparticles have a particle size of from about 1 to about 100 nanometers.

15. The method of any of claims 1 wherein the support material is chosen from silicon, a silicate such as a sodium silicate, a borosilicate, or a calcium aluminum silicates, clay, silicate, silica, starch, carbon, alumina, titania, calcium carbonate, barium carbonate, zirconia, metal oxide, carbon nanotubes, synthetic and natural zeolites, polymeric resins in bead or fibrous form, or mixtures of two or more thereof.

16. The method of any of claims 1 wherein the metal loading ranges from about 0.001 to 20 percent by weight of the support material.

17. The method of any of claims 1 wherein the metal loading ranges from about 0.05 to about 5 percent by weight of the support material.

18. The method of any of claims 1, wherein the support material comprises a functional group chosen from a group such as silanol, alkoxy, acetoxy, silazane, oximino-functional silyl group, hydroxyl, acyloxy, ketoximino, amine, aminoxy, alkylamide, hydrogen, allyl, an aliphatic olefinic group, aryl, hydrosulfide, or a combination of two or more thereof.

19. The method of any of claims 1, wherein the support material comprises a functional group chosen from —Si—CH═CH₂, —Si—OH, —Si—(CH₂)ₙC≡CH, —Si—(CH₂)ₙ—NH₂, —Si—(CH₂)ₙ—OH, —Si—(CH₂)ₙ—SH, or a combination of two or more thereof, and n is 1-26.

20. The method of any of claims 1 wherein the metal-containing polymer matrix is covalently bonded to the support material via a hydrophobic functional group attached to the support material.

21. The method of claim 20, wherein the hydrophobic group is chosen from an alkyldisilazane, a vinyl-containing silazane, or a combination thereof.

22. The method of any of claims 1, wherein the composition containing oil and water is at a temperature of from about 1° C. to about 1000° C.

23. The method of any of claims 1 further comprising:
applying a magnetic field to the water phase and removing the oil phase from the composition.

24. The method of claim 23, wherein the step of removing the oil phase comprises decanting the oil phase from the composition.

25. The method of claim 23 further comprising removing the supported magnetic particles from the water phase.

26. The method of claim 25 further comprising washing the supported magnetic particles that were removed from the water phase.

27. The method of claim 25 further comprising reusing the supported magnetic particles that were removed from the water phase in a subsequent operation to separate a composition comprising oil and water.

28. The method of any of claims 1, wherein the composition is a water-in-oil emulsion.

29. The method of any of claims 1, wherein the composition is an oil-in-water emulsion.

30. The method of any of claims 1, wherein the oil is chosen from a crude oil, a crude oil distillate, bitumen, a crude oil-light oil blend, a vegetable oil, an animal oil, a synthetic oil, or a combination of two or more thereof.

31. A solid demulsifier comprising:
functionalized magnetic metal nanoparticles covalently bonded to a support, wherein the functionalized magnetic nanoparticles comprise nanoparticles that exhibit magnetic properties, and said nanoparticles are encapsulated in a polymer matrix and/or are functionalized by stabilization of the nanoparticles with a C₇-C₃₀ organic fatty acid.

32. An emulsion comprising:
an oil,
water, and
a solid demulsifier, wherein the solid demulsifier comprises functionalized magnetic metal nanoparticles covalently bonded to a support, wherein the functionalized magnetic nanoparticles comprise nanoparticles that exhibit magnetic properties.

33. The emulsion of claim 32, wherein said nanoparticles are encapsulated in a polymer matrix and/or are functionalized by stabilization of the nanoparticles with a C₇-C₃₀ organic fatty acid.

34. The emulsion of claim 32, wherein the oil is chosen from a crude oil, a crude oil distillate, bitumen, a crude oil-light oil blend, a vegetable oil, an animal oil, a synthetic oil, or a combination of two or more thereof.
